(19) United States
(12) Patent Application Publication
(10) Pub. No.: US 2024/0068583 A1
(43) Pub. Date: Feb. 29, 2024

(54) LOW PRESSURE VALVE ASSEMBLIES

(71) Applicant: Novoheart International Limited, Kowloon (HK)

(72) Inventors: Yosuke Kurokawa, Irvine, CA (US); Eugene Lee, Irvine, CA (US)

(21) Appl. No.: 18/260,754

(22) PCT Filed: Jan. 7, 2022
(86) PCT No.: PCT/US2022/011671

§ 371 Date: Jul. 7, 2023

Related U.S. Application Data
(60) Provisional application No. 63/135,529, filed on Jan. 8, 2021.

(51) Int. Cl. F16K 15/14; F16K 15/16; F16K 99/00

(52) U.S. Cl. CPC F16K 15/1402; F16K 15/147; F16K 15/16; F16K 99/0001

(57) ABSTRACT

A low pressure valve assembly comprising: an elastomeric material including at least one cut that forms a deformable portion, a first backing material including an inlet, and a second backing material including an outlet, wherein the elastomeric material is between the first backing material and the second backing material, and wherein the deformable portion is configured to deform at a range of pressures including a pressure of less than or equal to about 1 mmH20.

### CROSS REFERENCE TO RELATED APPLICATION

This application claims the benefit of U.S. provisional patent application No. 63/135,529, filed Jan. 8, 2021, the entire disclosure of which is incorporated herein by reference.

### FIELD

Described herein are low pressure valve assemblies.

### SUMMARY

Described herein are valves that can actuate at low pressures while preventing flow in the reverse direction. Although these valves can actuate at low pressures, they still retain the ability to actuate at higher pressures as well. The valves can include a thin, elastomeric material that comprises at least two cuts and is layered between two backings. The backings can control directionality of valve opening.

In some embodiments, low pressure valve assemblies are described. These valve assemblies can include an elastomeric material including at least one cut, and in some embodiments at least two cuts, that forms a deformable portion, a first backing material including an inlet, and a second backing material including an outlet. In some embodiments, the elastomeric material is between the first backing material and the second backing material.

In some embodiments, the valve assemblies are configured to function and/or activate at a range of pressures including a pressure of less than or equal to about 1 mmH₂O. This function can be a result of movement or reconfiguration of the deformable portion(s). In some embodiments, the deformable portion is configured to deform at a pressure of less than or equal to about 1 mmH₂O. In some embodiments, the deformable portion can also deform at higher pressures, but can still retain functionality at lower pressures. In other embodiments, the deformable portion is configured to deform at a pressure of less than or equal to about 1 mm H₂O.

Embodiments include configurations where the outlet is circular and/or is configured to not obstruct the deformable portion.

Other embodiments include configurations where the inlet is rectangular and/or is configured to obstruct the deformable portion and/or prevent backflow.

In some embodiments, the at least two cuts are parallel.

Some valve assemblies described herein include elastomeric materials that include at least one relief cut, or four relief cuts. The relief cuts in some embodiments are not exposed beyond the first backing material and the second backing material.

Further described herein are low pressure valve assemblies comprising an elastomeric material including at least one cut, and in some embodiments at least two cuts that form a first deformable portion and at least one cut, and in some embodiments at least two second cuts that form a second deformable portion, a first backing material including a first inlet and a first outlet, and a second backing material including a second inlet and a second outlet. In some embodiments, the elastomeric material is between the first backing material and the second backing material. In some embodiments, the first deformable portion and the second deformable portion are configured to deform at a pressure range including pressures of less than or equal to about 1 mmH₂O. In some embodiments, the deformable portions can also deform at higher pressures, but can retain functionality at lower pressures. In other embodiments, the first deformable portion and the second deformable portion are each configured to deform at a pressure of less than or equal to about 1 mm H₂O.

In some embodiments, the first outlet is circular, the second outlet is circular, or the first and second outlets are circular. In some embodiments, the first outlet and the second outlet are each configured to not obstruct the first deformable portion or the second deformable portion.

In some embodiments, the first inlet is rectangular, the second inlet is rectangular, or the first and second inlets are rectangular. In some embodiments, the first inlet and the second inlet are each configured to obstruct the first deformable portion and the second deformable portion and/or are each configured to prevent backflow.

In some embodiments, the elastomeric material further includes at least one relief cut for each of the first deformable portion and the second deformable portion.

The valve assemblies as described herein can be used to pump fluid at a low pressure.

### BRIEF DESCRIPTION OF THE DRAWINGS

FIGS. 1A-1C illustrate an embodiment of a valve as described herein. FIG. 1A illustrates the valve in the resting state. FIG. 1B illustrates the valve in an open state with head pressure. FIG. 1C illustrates the valve closed and experiencing back pressure.

FIG. 2 illustrates an elastomeric material portion with the deformable region in an extended or open state.

FIG. 3 is an image of the valve under a microscope.

FIGS. 4A-4C illustrate a dual valve configuration. FIG. 4A illustrates the elastomeric material including two deformable regions. FIG. 4B illustrates the elastomeric material from FIG. 4A sandwiched between two backing material portions. The valve assembly illustrated in FIG. 4B has the valves configured in opposite directions. FIG. 4C is an exploded view of the assembly of FIG. 4B.

FIGS. 5A-5C illustrate another dual valve configuration. FIG. 5A illustrates the elastomeric material including two deformable regions. FIG. 5B illustrates the elastomeric material from FIG. 5A sandwiched between two backing material portions. The valve assembly illustrated in FIG. 5B has the valves configured in opposite directions. FIG. 5C is an exploded view of the assembly of FIG. 5B.

FIG. 6 illustrates an elastomeric material that includes two deformable regions each surrounded by additional relief cuts.

FIG. 7 illustrates an elastomeric material that includes two deformable regions each with curved, or non-linear cuts.

FIG. 8 illustrates an elastomeric material that includes two deformable regions each with a single cut forming each deformable region.

FIG. 9 illustrates another elastomeric material that includes two deformable regions each with a single cut forming each deformable region.

### DETAILED DESCRIPTION

Described herein are valves and valve assemblies used for systems and flow channels with low, and in some instances very low pressure. Low pressure as used herein generally refers to pressures of about or less than about 1 mmH₂O. In some embodiments, the valves described herein can function throughout a pressure range and that pressure range can include the low pressures described herein as well as higher pressures. Low pressure can include pressure ranges that include a pressure as low as or lower than about 1 mmH₂O.

Commonly, check valves are used in various flow systems. Check valves generally have two openings and only allow flow in a single direction. One type of check valve is a diaphragm check valve, which uses a rubber or plastic diaphragm to control flow through the valve. A diaphragm check valve relies on back pressure to seal a diaphragm and opens when sufficient pressure moves or deflects the diaphragm to allow fluid through the valve.

However, existing check valves often require high actuation pressures when configured as normally closed and/or a high closing pressure when configured as normally open. Theses valves will not function at target pressures of the valves described herein, for example, about or less than about 1 mmH₂O. In some embodiments, the valves described herein can work at the low pressures described, but also at higher pressures.

One area where low pressure valves are required is in biological systems, such as, but not limited to the heart. Valves described herein can be used with in vitro heart systems such as a human ventricular Cardiac Organoid Chamber (hvCOC). These types of devices are macroscopic human cardiac tissue models that are capable of mimicking fluid pumping similar to a natural heart that can be used for many in vitro purposes. These purposes can be assessing cardiac toxicity screening, performing disease modeling, performing regenerative studies, and performing functional assessments.

In such systems, valves can be used to control fluid flow from an inlet towards an outlet. This enables independent control of preload and afterload (by applying differing pressures at the inlet and outlet, respectively), and can also result in more physiological pressure-volume loops.

In some embodiments, the valves as described herein can actuate at pressures of about or less than about 1 mmH₂O. In some embodiments, the valves can actuate across a range of pressures that includes the low pressures described herein as well as at higher pressures.

In some embodiments, the valves as described herein can prevent backflow.

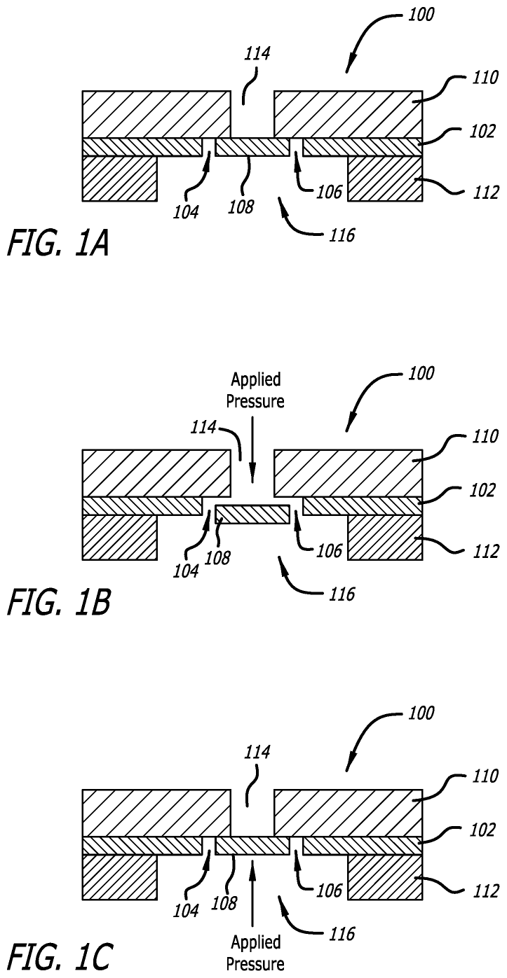

As illustrated in FIG. 1A, valve 100 includes a layer of thin elastomeric material 102. Elastomeric material 102 can include at least two cuts, such as first cut 104 and second cut 106 thereby creating a deformable region 108. In some embodiments, these cuts are referred to as vertical cuts. However, the cuts can be included in any orientation that allows for valve function.

Elastomeric material 102 resides between a first backing material 110 and a second backing material 112. First backing material 110 can include an inlet 114 and second backing material 112 can include an outlet 116. In some embodiments, however, first backing material 110 can include an outlet and second backing material 112 can include an inlet.

As illustrated in FIG. 1B, when pressure is applied at inlet 114, deformable region 108 deflects in response to that applied pressure. Outlet 116 is large enough to enable deformable region 108 to expand freely. When the pressure applied at inlet 114 is removed, deformable region 108 reverts back to its natural resting position in line with the remainder of elastomeric material 102.

As illustrated in FIG. 1C, when pressure is applied at outlet 116, deformable region 108 attempts to deflect in response to that applied pressure. However, inlet 114 is not large enough to enable deformable region 108 to expand. Thus, deformable region 108 cannot move in response to this applied pressure, thereby enabling the valve to prevent backflow.

Valve 100 is closed at rest due to adhesion of the elastomeric material to one side of the backing.

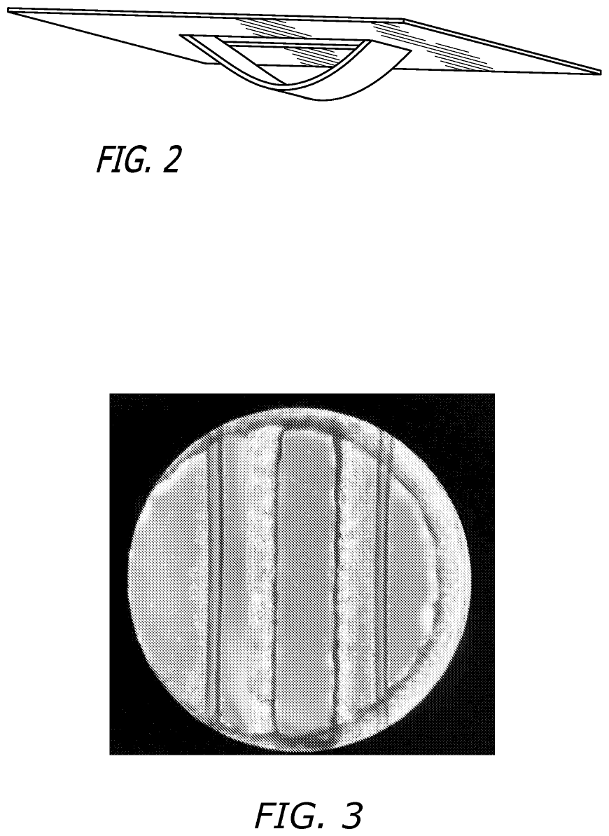

FIG. 2 shows a 3D model of the elastomeric material layer in an exaggerated, deflected position.

FIG. 3 is an image of the valve under a microscope.

The dimensions of the cuts described herein can be modified to achieve different flow characteristics. In some embodiments, a wider cut can increase the overlapping area between the elastomeric material and the backing to increase actuation pressure. In some embodiments, the opening width on the first or second backing material can be decreased to achieve a similar increase in actuation pressure.

In some embodiments, an elastomeric layer can include two or more sets of cuts and first and second backing materials can have two or more inlets/outlets each. For example, in one embodiment, an elastomeric material can include two sets of cuts thereby forming two different deformable regions in the elastomeric material. If two valves are intended to flow in the same direction, first backing material can include two inlets and second backing material can include two outlets. Likewise, first backing material can include two outlets and second backing material can include two inlets.

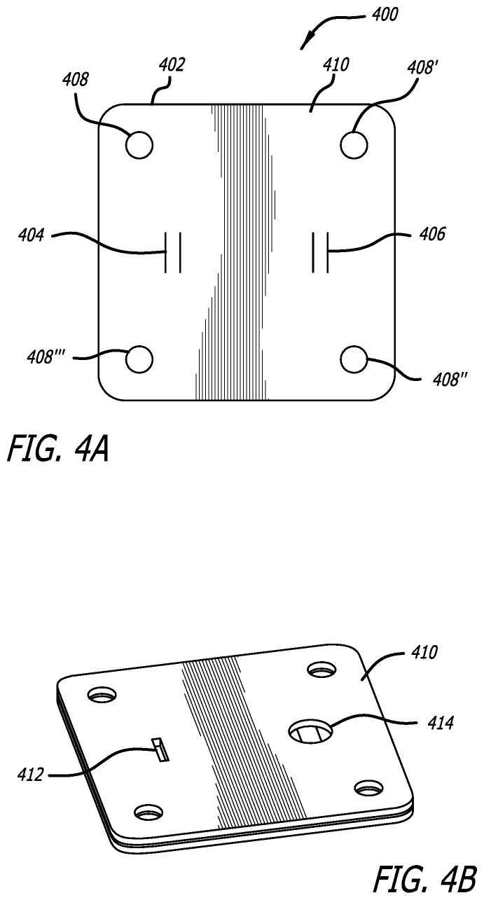

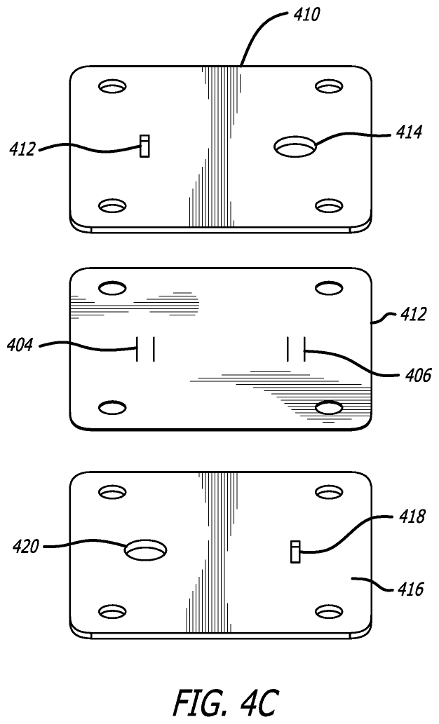

If on the other hand, two opposite valves are desired, first backing material can include one inlet and one outlet and second backing material can include one inlet and one outlet. Such a configuration is illustrated in FIG. 4A-4C. Therein, elastomeric material 402 includes first deformable region 404 and second deformable region 406. Mounting holes 408, 408′, 408″, 408′″ can be used to install valve assembly 400 at a desired location. These mounting holes can be located anywhere in the elastomeric material and accompanying backing materials that do not interfere with the deformable region(s).

In embodiments herein, mounting holes or equipment mounting holes can be any shape and/or size that accommodate mounting, orientation, convenience, pass-through, access, or the like. Shapes can include, but are not limited to circular, square, elliptical, triangular, rectangular, or any other rectilinear or circular shape.

Likewise, first backing material 410 can include inlet 412 and outlet 414, and second backing material 416 can include inlet 418 and outlet 420. First backing material 410 and second backing material 416 can include mounting holes that align with those in elastomeric material 402.

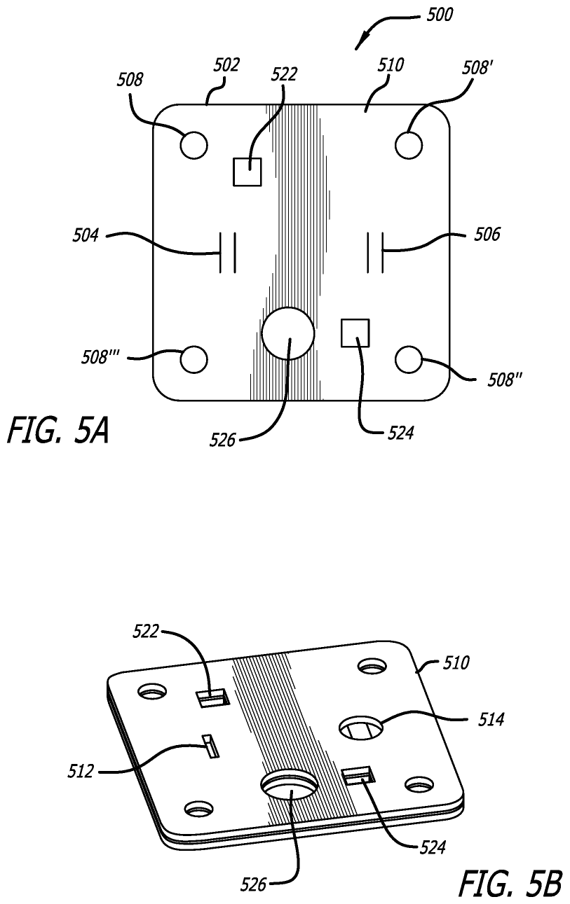

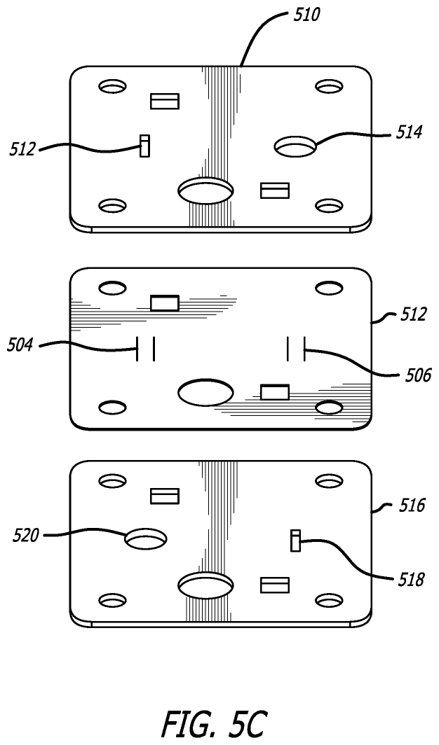

Another configuration of a valve assembly, valve assembly 500, including two opposite valves is illustrated in FIG. 5A-5C. Therein, elastomeric material 502 includes first deformable region 504 and second deformable region 506. Mounting holes 508, 508′, 508″, 508′″ can be used to install valve assembly 500 at a desired location. Valve assembly 500 further includes one or more additional equipment mounting hole(s) 522, 524, 526. Both mounting holes and equipment mounting holes can be located anywhere in the elastomeric material and accompanying backing materials that do not interfere with the deformable region(s).

Likewise, first backing material 510 can include inlet 512 and outlet 514, and second backing material 516 can include inlet 518 and outlet 520. First backing material 510 and second backing material 516 can include mounting holes that align with those in elastomeric material 502.

In embodiments herein, mounting holes or equipment mounting holes can be used to mount the herein described valves and/or valve assemblies into a human ventricular Cardiac Organoid Chamber (hvCOC) device.

In some embodiments, elastomeric material can include one or more additional cut(s) in the region of the at least two cuts that form the deformable region. These additional cut(s) can be referred to as relief cuts and can, in some embodiments, increase valve consistency. By adding relief cuts around the deformable region, there is a lower chance of pretension in the deformable region that can prevent the valve from normally closing. The relief cuts also add some flexibility in the elastomer that can assist the alignment process with the backings during valve assembly.

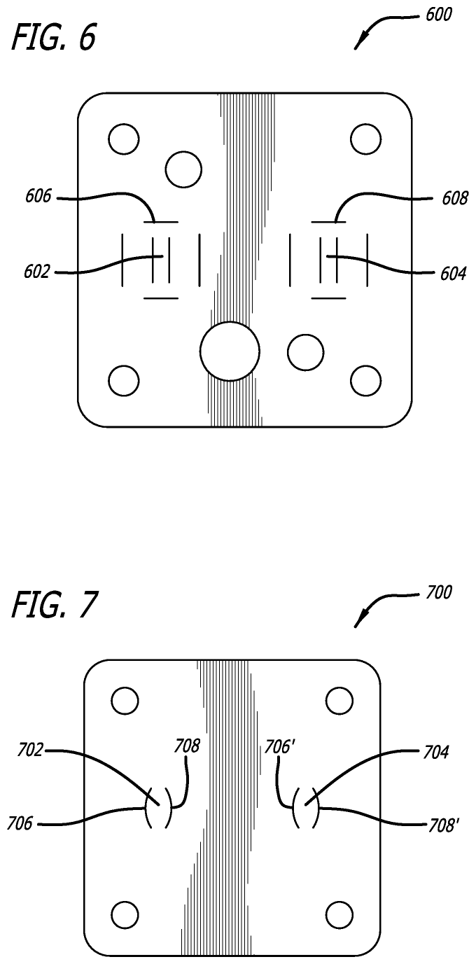

FIG. 6 illustrates elastomeric material 600 that includes first deformable region 602 and second deformable region 604. Elastomeric material 600 can also include at least one additional relief cut 606 and 608 near first deformable region 602 and second deformable region 604 respectively. In the FIG. 6 embodiment, each of first deformable region 602 and second deformable region 604 includes four relief cuts each perpendicular to its neighbor.

However, in other embodiments, each deformable region can include one, two, three, four, five, six, seven, eight, nine, ten or more relief cuts. These relief cuts can be end to end to form a surrounding configuration or each cut outward forming a radially outward configuration.

Each relief cut described herein is completely enclosed between first and second backing materials thereby preventing leaking through these relief cuts.

FIG. 7 illustrates elastomeric material 700 that includes first deformable region 702 and second deformable region 704. First deformable region 702 and second deformable region 704 can each independently include two cuts, such as first cut 706, 706′ and second cut 708, 708.′ In the embodiment of elastomeric material 700, first cut 706, 706′ and second cut 708, 708′ are curved cuts that are oriented away from one another. In another embodiment, the curved cuts can be toward one another or face the same direction. As described, cuts can be of any shape that allow valve function at the desired pressure or pressure range.

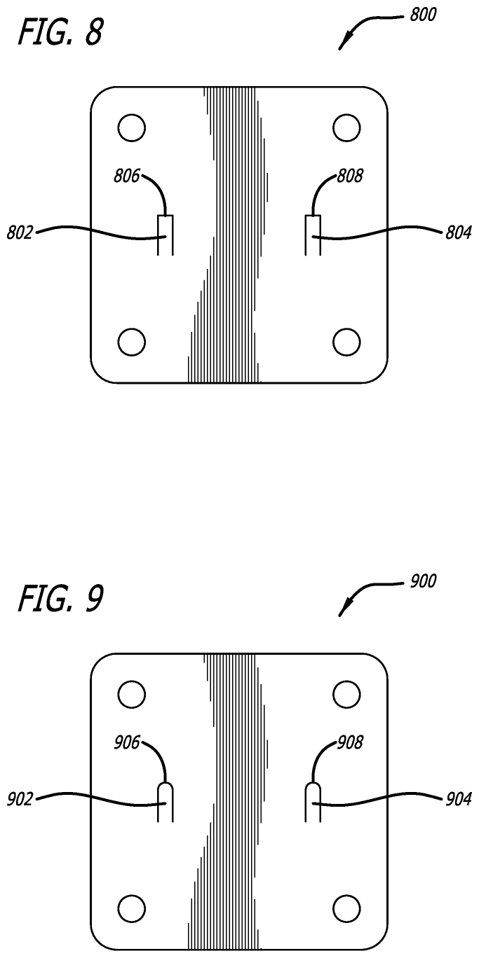

FIG. 8 illustrates elastomeric material 800 that includes first deformable region 802 and second deformable region 804. First deformable region 802 and second deformable region 804 can each independently include one cut, such as cut 806 and cut 808. In the embodiment of elastomeric material 800, each cut is a single cut that provides a flap of material than can deform as described herein. First deformable region 802 and second deformable region 804 include two parallel portions and a perpendicular end portion.

However, other single cut configurations are possible such as, but not limited to non-parallel portions that meet at a common point (e.g. triangular), which can be a point, curve, rectilinear edge, or the like. In another embodiment, a single cut configuration can include nonparallel curved portions and meet at a common point or are connected through another portion.

FIG. 9 illustrates elastomeric material 900 that includes first deformable region 902 and second deformable region 904. First deformable region 902 and second deformable region 904 can each independently include one cut, such as cut 906 and cut 908. In the embodiment of elastomeric material 900, each cut is a single cut that provides a flap of material than can deform as described herein. First deformable region 902 and second deformable region 904 include two parallel portions and curved end portion connecting the two parallel portions.

In some embodiments, deformable portions when including a single cut can be any shape that allows the valves to function at the pressures described herein.

All three layers (elastomer+two backings) can be cut from bulk sheets of the material. Cutting can be by any viable technique. In one embodiment, that technique is laser cutter. However, in some embodiments, die-punching can also be used. Laser cutting can be very rapid and easy to scale in production. Laser cutting also allows rapid changes in the design as necessary, whereas die-cutting requires tooling a new die if a design change is made.

Further, in some embodiments, no advanced fabrication equipment is necessary for the assembly of the valves described herein.

### Example 1

A valve assembly as described herein with two valves, such as that illustrated in FIGS. 4A-4C is installed into a hvCOC device to assist in fluid flow between chambers. The inter-chamber pressure does not exceed 1 mmH₂O. No backflow is experienced. In fact, when inter-chamber pressure does not exceed 1 mmH₂O, neither forward nor back flow is experienced. However, once the pressure exceeds about 1 mmH₂O, forward flow is observed, but backflow is still not observed.

### Example 2

A valve assembly as described herein with two valves, such as that illustrated in FIGS. 5A-5C, is installed into a hvCOC device to assist in fluid flow between chambers. The inter-chamber pressure does not exceed 1 mmH₂O. No backflow is experienced. In fact, when inter-chamber pressure does not exceed 1 mmH₂O, neither forward nor back flow is experienced. However, once the pressure exceeds about 1 mmH₂O, forward flow is observed, but backflow is still not observed.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A low pressure valve assembly comprising:
an elastomeric material including at least one cut that forms a deformable portion,
a first backing material including an inlet, and
a second backing material including an outlet,
wherein the elastomeric material is between the first backing material and the second backing material, and
wherein the deformable portion is configured to deform at a range of pressures including a pressure of less than or equal to about 1 mmH₂O.

2. The low pressure valve assembly of claim 1, wherein the elastomeric material includes at least two cuts.

3. The low pressure valve assembly of claim 1, wherein the outlet is circular.

4. The low pressure valve assembly of claim 1, wherein the outlet is configured to not obstruct the deformable portion.

5. The low pressure valve assembly of claim 1, wherein the inlet is rectangular.

6. The low pressure valve assembly of claim 1, wherein the inlet is configured to obstruct the deformable portion.

7. The low pressure valve assembly of claim 6, wherein the inlet is configured to prevent backflow.

8. The low pressure valve assembly of claim 1, wherein the at least two cuts are parallel.

9. The low pressure valve assembly of claim 1, wherein the elastomeric material further includes at least one relief cut.

10. The low pressure valve assembly of claim 9, wherein the elastomeric material further includes four relief cuts.

11. A low pressure valve assembly comprising:
an elastomeric material including at least one first cut that forms a first deformable portion and at least one second cut that forms a second deformable portion,
a first backing material including a first inlet and a first outlet, and
a second backing material including a second inlet and a second outlet,
wherein the elastomeric material is between the first backing material and the second backing material, and
wherein the first deformable portion and the second deformable portion are each configured to deform at a range of pressures including a pressure of less than or equal to about 1 mmH₂O.

12. The low pressure valve assembly of claim 11, wherein the elastomeric material includes at least two cuts.

13. The low pressure valve assembly of claim 11, wherein the first outlet is circular.

14. The low pressure valve assembly of claim 11, wherein the second outlet is circular.

15. The low pressure valve assembly of claim 11, wherein the first outlet and the second outlet are each configured to not obstruct the first deformable portion or the second deformable portion.

16. The low pressure valve assembly of claim 11, wherein the first inlet is rectangular.

17. The low pressure valve assembly of claim 11, wherein the second inlet is rectangular.

18. The low pressure valve assembly of claim 11, wherein the first inlet and the second inlet are each configured to obstruct the first deformable portion and the second deformable portion.

19. The low pressure valve assembly of claim 18, wherein the first inlet and the second inlet are each configured to prevent backflow.

20. The low pressure valve assembly of claim 11, wherein the elastomeric material further includes at least one relief cut for each of the first deformable portion and the second deformable portion.

21. A method of pumping fluid using a valve assembly of claim 1.
